(19) United States
(12) Patent Application Publication
(10) Pub. No.: US 2007/0269831 A1
(43) Pub. Date: Nov. 22, 2007

(54) Method for Early Detection of Ovarian Cancer

(75) Inventors: David Spriggs, New York, NY (US); Jakob Dupont, Pelham, NY (US)

Correspondence Address: Marina Larson & Associates, LLC, P.O. BOX 4928, DILLON, CO, 80435, US

(73) Assignee: SLOAN-KETTERING INSTITUTE FOR CANCER RESEARCH, New York, NY (US)

(21) Appl. No.: 10/595,135

(22) PCT Filed: Sep. 10, 2004
(86) PCT No.: PCT/US04/29902

§ 371 Date: Apr. 6, 2007

Related U.S. Application Data
(60) Provisional application No. 60/502,160, filed on Sep. 10, 2003.

(51) Int. Cl. G01N 33/574

(52) U.S. Cl. 435/7.1; 435/7.23

(57) ABSTRACT

YKL-40 levels in serum and plasma samples provide as an indicator of both the presence of early stage ovarian cancer and of its aggressiveness. Thus testing for YKL-40 levels in patients at risk but not yet displaying symptoms associated with ovarian cancer, or in patients newly diagnosed with early stage ovarian cancer (Stage I or II) enhance both the detection of early stage ovarian cancer and selection of treatment protocols used for early stage ovarian cancer patients.

This application claims the benefit under 35 USC § 119(e) of U.S. Provisional Application 60/502,160 filed Sep. 10, 2004, which application is incorporated herein by reference in its entirety.

This application was developed with the support of NIH Grant No. PO1 CA 52477-12. The United States government may have certain rights in this invention.

### BACKGROUND OF THE INVENTION

The present application relates to a method for detection of ovarian cancer and in particular a method for screening of individuals who may have a recognized increased risk of ovarian cancer but who have not previously been identified as having ovarian cancer. The method of the invention is particularly suitable because it provides better detection for early stage ovarian cancer.

Ovarian cancer is the leading cause of death from gynecologic malignancies in the United States. Over 23,000 cases are diagnosed yearly, and there are an estimated 14,000 deaths per year due to ovarian cancer. More than 70% of patients have stage III or IV disease at the time of diagnosis. Despite the introduction of new chemotherapeutic agents for the treatment of ovarian cancer, the fact that most patients are diagnosed with advanced-stage disease translates into a poor 5-year survival of 20% to 30%. On the other hand, 90% of women diagnosed with disease confined to the ovary survive more than 5 years. Thus, early detection of ovarian cancer is essential for improved survival. The development of serum-based diagnostic tests for the detection of early-stage cancers in asymptomatic patients has been an important endeavor. Unlike breast or prostate cancer, there is no simple diagnostic test to detect early-stage ovarian tumors. Despite considerable efforts, no cost-effective screening tests have been developed.

Currently, a number of ovarian cancer markers have been defined. These include: CA125, CA15-3, carcinoembryonic antigen (CEA), the kallikrein family (including hK13, hK10, and hK6), prostasin, lysophosphatidic acid (LPA), and others. CA125 was the first tumor marker available for the management of ovarian cancer and remains the best. Elevation of CA125 in the serum can be detected in benign conditions, including pregnancy, endometriosis, ovarian cysts, and cirrhosis, and in malignant conditions, such as ovarian, fallopian tube, primary peritoneal, cervical, endometrial, breast, colon, and lung cancers. Approximately 80% of patients with epithelial ovarian cancer have elevations of CA125. CA125 is useful for disease monitoring, assessing response to therapy, and the detection of relapse. The major problems with CA125 serum marker include poor sensitivity and specificity for ovarian cancer, especially for the diagnosis of early-stage disease. CA125 is elevated in only 40% to 50% of patients with stage I/II tumors.

CA15-3 is elevated in several tumors including breast cancer, and it is elevated in approximately 70% of epithelial ovarian cancer patients, predominantly in those with advanced disease.

The human kallikrein gene family is a subfamily of serine proteases and can be involved in the progression and metastasis of human cancers. Recent studies have detected the secreted kallikreins 6 and 10 in patients with ovarian cancer.

Prostasin has recently been evaluated as another serum marker for non-mucinous ovarian carcinomas, as has LPA. In a small study, plasma LPA concentrations were elevated in 90% of women with stage I disease and 100% of women with advanced and recurrent disease compared with healthy controls. The current method of measuring LPA, however, which involves lipid extraction followed by gas chromatography, may limit its utility.

Accordingly, not withstanding these existing markers, there remains a need for a screening test that can be used to identify early stage ovarian cancer in patients. It is an object of the present invention to provide such a test.

### SUMMARY OF THE INVENTION

The present invention provides a method for detection of ovarian cancer in an individual, comprising the steps of

(a) obtaining a sample from the individual;

(b) determining the amount of expressed YKL-40 in the sample; and

(c) comparing the amount of expressed YKL-40 determined to a pre-determined threshold, wherein if the predetermined threshold is exceeded, the test is deemed to be an indicator of ovarian cancer in the individual. The method is particularly applicable for screening individuals who have increased risk factors for ovarian cancer, for example a parent or sibling with a prior diagnosis of ovarian cancer, but who are not themselves evidencing overt symptoms of ovarian cancer of the type associated with Stage III or IV ovarian cancer. By testing such individuals, the test of the invention allows the early detection of ovarian cancer, thus facilitating early treatment and an improved long term prognosis.

The present invention also provides a method for assessing the risk of post-treatment recurrence in a patient diagnosed with early stage ovarian cancer comprising the steps of

(a) obtaining a pre-operative sample from the individual and

(b) determining the amount of expressed YKL-40 in the sample. A pre-operative level of greater than 80 ng/mL is indicative of an increased risk of post-treatment recurrence of ovarian cancer.

### BRIEF DESCRIPTION OF THE FIGURES

FIG. 1: Box plots comparing YKL-40 values in preoperative ovarian cancer patients with those of normal individuals, individuals with benign gynecologic processes, and high-risk screening individuals with or without prior breast cancer. Values for one ovarian cancer patient and two normal controls with rheumatoid arthritis are indicated by “x”; these values were eliminated from statistical comparisons.

FIGS. 2 A-D: Preoperative YKL-40 values considering tumor stage (A), grade (B), histological diagnosis (C), and age (D). All plots are on logarithmic scale. Vertical lines show log-scale mean (marked by cross-bar) +/−2 standard deviations. Plot for age includes a curve representing a cubic spline trend estimate.

FIGS. 3A-C: ROC curves of YKL-40 and CA125 in ovarian cancer compared with high-risk screening (A), normal (B), or benign gynecologic disease (C) patients.

FIG. 4: Time to recurrence of stage I and II ovarian cancer patients considering preoperative elevation level of serum YKL-40. Two groups were compared: patients with YKL-40 values <80 ng/mL and >80 ng/mL.

### DETAILED DESCRIPTION OF THE INVENTION

The present invention provides a method for detection of ovarian cancer in an individual based on an assessment of the amount of expression of YKL-40. YKL-40 is a glycoprotein in the chitinase protein family. The gene for YKL-40 is located on chromosome 1q32 (Renkema, et al. J Biol Chem (1985) 270:2198-2202) and is a mammalian member of the 18-glycosyl-hydrolase family (Renkema et al., Eur. J. Biochem. (1998) 251: 504-509), a gene family that includes bacterial and fungal chitinases. YKL-40 has significant sequence similarity to the chitin-degrading enzyme, chitinase (Kurose et al., Am. J. Pathol. (2001) 158: 2097-2106); it has been shown to bind chitin, but retains no chitinase activity and has not been determined to have any other enzymatic activity or function. The full spectrum of mammalian polysaccharide structures that bind to YKL-40 and the function of this protein are unknown.

YKL-40 has been evaluated as a serum marker for conditions such as rheumatoid arthritis (Register et al. Clin. Chem. (2001) 47: 2159-2161), severe osteoarthritis (Johansen et al., Eur. J. Cancer (1995) 31: 1437-1442), hepatic fibrosis, primary colorectal cancer (Cintin et al., Br. J. Cancer (1999) 79: 1494-1499), glioblastoma (Matsumoto et al., Clin. Exp. Rheumatol. (2001) 19: 655-660), metastatic breast cancer (Henrissat et al., Biochem. J. (1993) 293: 781-788), and recurrent ovarian cancer (Dehn et al., Acta Obstet Gynecol Scand (2003) 82: 287-293). These data suggest YKL-40 may be involved in extracellular matrix degradation and/or angiogenesis (Johansen et al., J. Bone Miner. Res. (1992) 7: 501-512; DeLong et al., Biometrics (1988) 44: 837-845).

What has not been appreciated before the present invention, however, is the usefulness of YKL-40 as an indicator of both the presence of early stage ovarian cancer and of its aggressiveness. The invention of this application is thus a method that exploits this discovery to enhance both the detection and treatment protocols used for early stage ovarian cancer patients.

In one embodiment of the invention, a method is provided for detecting the presence of early stage ovarian cancer in an individual. When using the method as a screening test, the individual to be tested is female, and is one who has not been diagnosed with ovarian cancer and who is not displaying symptoms associated with stage III or IV ovarian cancer. Such symptoms include general abdominal discomfort and/or pain (gas, indigestion, pressure, swelling, bloating, cramps, etc.), nausea, diarrhea, constipation, or frequent urination, loss of appetite, feeling of fullness even after a light meal, weight gain or loss with no known reason and abnormal bleeding from the vagina. In a second embodiment of the invention, a method is provided for assessing the aggressiveness of ovarian cancer in an individual diagnosed with stage I or stage II ovarian cancer.

In each of these embodiments, the first step of the method is obtaining a sample from the individual for testing. Suitable samples are serum or plasma samples.

The sample is then tested for the amount of expressed YKL-40 that is present. The methodology employed for detection and quantification of YKL-40 is not critical provided it supplies the type of reliability and quantitative accuracy appropriate for use in a diagnostic assay. Such methods include, without limitation, radio immunoassay, enzyme immunoassay, and ELISA. U.S. Pat. No. 6,579,684, which is incorporated herein by reference, discloses the use of YKL-40 as a marker and prognostic indicator of cancer. Various methods are disclosed in this publication for the detection of YKL-40, including in particular immunoassays using radio and other labels types, and each of these assays may be employed in the present invention. In addition, an enzyme-immunoassay for serum YKL-40 is commercially available from Quidel Corporation. San Diego, Calif.

Once the amount of YKL-40 in the sample is determined, this amount is compared to a pre-determined threshold. The pre-determined threshold is set based on “normal” values detected in individuals without ovarian cancer at a level such that determined YKL-40 values greater than the pre-determined threshold are indicative of the presence of early stage ovarian cancer. The selection of a particular threshold value for diagnostic testing purposes reflects a balancing of the desire to exclude false positives, while at the same time identifying substantially all individuals who have ovarian cancer. In the examples included in this application, the threshold used was one which was two standard deviations above the mean value detected in normal individuals, i.e, ≧40 ng/mL, for example ≧61 ng/mL. Such a value will result in very few false positive, and was found to identify 72% of ovarian tumors, and 65% of the individuals with stage 1 and 2 tumors, numbers substantially better than those achieved with CA-125 or Ca15-3, the currently established markers for ovarian cancer. It will be appreciated that selection of a lower threshold captures more early stage tumors, albeit at the price of some false positives. The threshold level may be derived from a general population, or it may be a more specific threshold level derived from “normal” individuals in a subpopulation (for example based on age or other criteria) of which the tested individual is a member, including populations at high risk for developing ovarian cancer.

The determination of levels of YKL-40 above the threshold value is a valid indicator of ovarian cancer. However, a final diagnosis of ovarian cancer will generally be made only after follow-up confirmatory testing using other methods, for example more expensive and/or more invasive tests including diagnostic imaging (CT Scan, MRI, PET scan, etc), and other appropriate steps selected by the treating physician are then performed to arrive at an actual diagnosis.

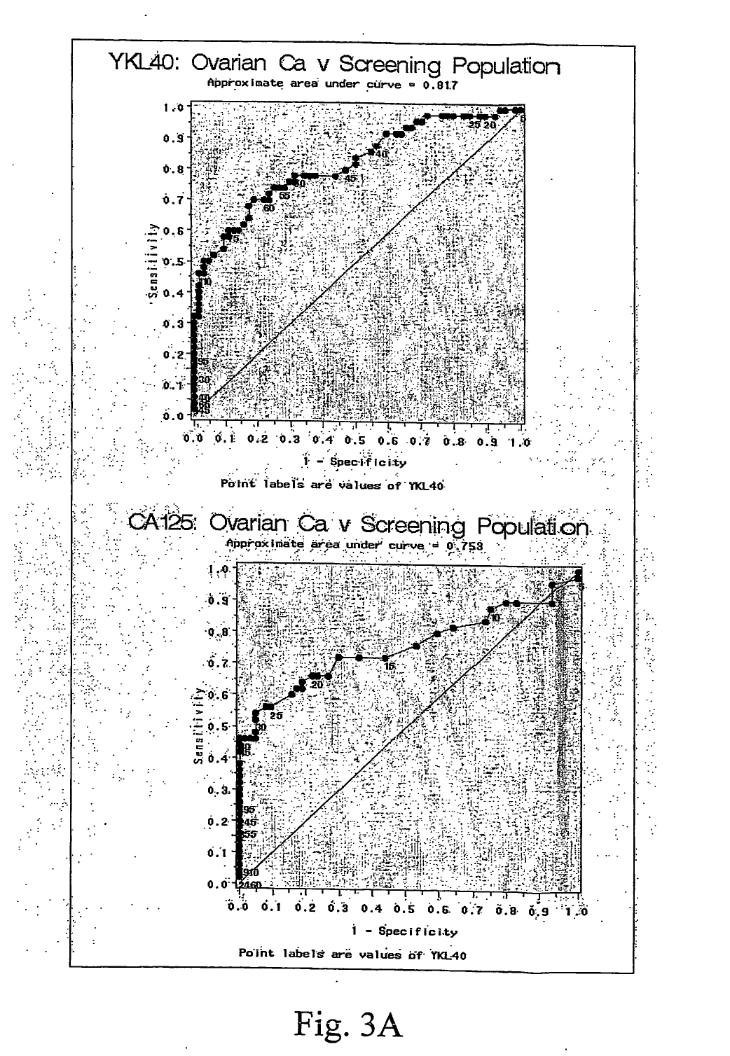

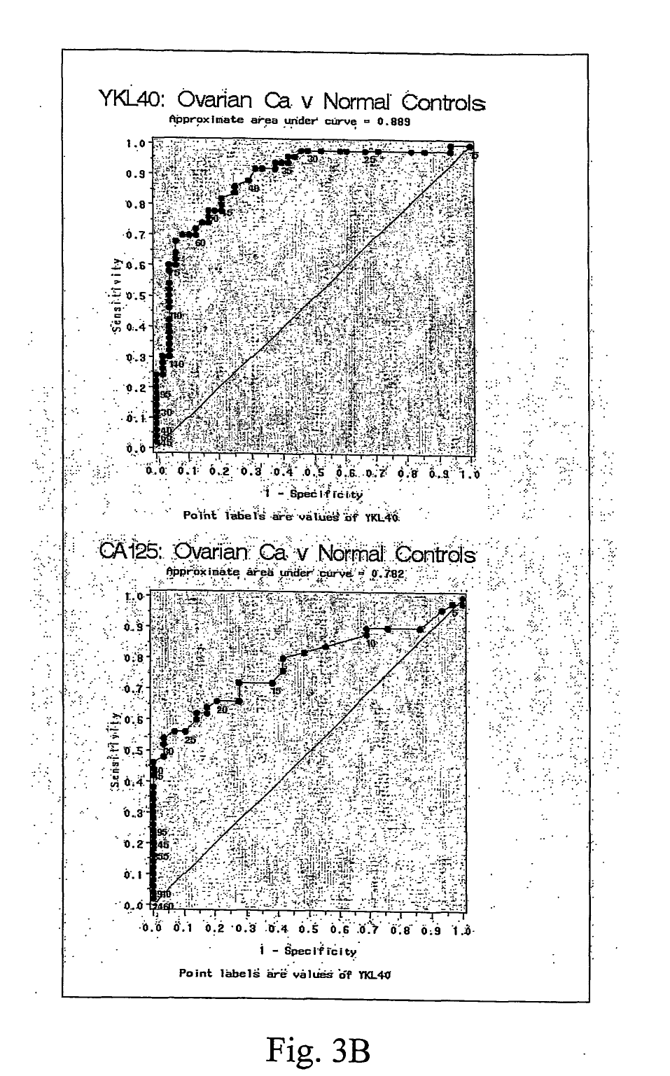

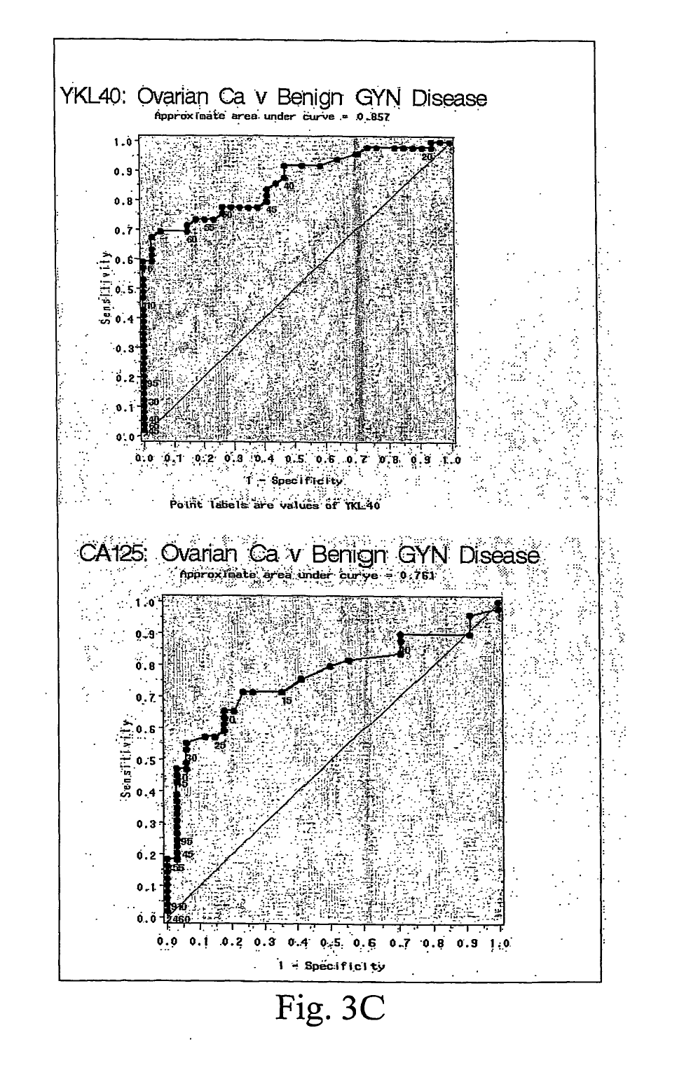

Tests for YKL-40 levels can also be used in combination with other marker-screening tests, such as tests for CA-125 or CA15-3. The ROC curves (FIG. 3) presented reveal that YKL-40 has a sensitivity of 72% and a specificity of 90% for the prediction of ovarian cancer in this study. The sensitivity and specificity values for CA125 for the detection of ovarian cancer were 46% and 95%, respectively. Serum YKL-40 assessment can be combined with other tumor markers, such as CA125 and CA15-3, to increase the sensitivity and specificity of ovarian cancer detection. Using all three markers, it was possible to detect 74% of early-stage tumors.

When a diagnosis of early stage ovarian cancer is made, regardless of whether an initial screening is performed as above, or whether the diagnosis is made using other methods entirely, pre-operative YKL-40 levels in patients diagnosed with early stage ovarian cancer can be used to identify patients who are at high risk for disease recurrence. In particular, patients with pre-operative YKL-40 values greater than 80 ng/mL have a 71% recurrence rate versus patients with YKL-40 value less than 80 ng/mL, who had a 0% recurrence rate. Furthermore, 64% of patients with YKL-40 values >80 ng/1 mL died of disease, while none of the patients with lower preoperative YKL-40 values died. Thus, YKL-40 can be used to identify high risk ovarian cancer patients that might require more aggressive treatment and follow-up. Preoperative levels of CA125 and CA15-3 in these patients did not correlate with a poor outcome. Thus, YKL-40 can identify early-stage patients who are at high risk for recurrence and disease-related death, and this information can be used to guide treatment decisions.

In a further aspect, the invention therefore provides a method for assessing the risk of post-treatment recurrence in a patient diagnosed with early stage ovarian cancer comprising the steps of obtaining a pre-operative sample from the individual and determining the amount of expressed YKL-40 in the sample; wherein a pre-operative level of greater than 80 ng/mL is indicative of an increased risk of post-treatment recurrence of ovarian cancer.

The invention will now be further illustrated and described with reference to the following non-limiting examples.

Materials and Methods

Study Population

Patients undergoing gynecologic oncologic surgery at Memorial Sloan-Kettering Cancer Center (MSKCC) have their tumor specimens and serum samples banked under an Institutional Review Board- (IRB-) approved tissue-acquisition protocol after signing informed consent. MSKCC pathologists reviewed all tumor specimens. Patients in the study were operated on between 1992 and 2002. Patients were selected based upon a diagnosis of ovarian, fallopian tube, or primary peritoneal cancer and were preferentially selected if they were diagnosed with an early-stage cancer. In addition, the first 31 patients identified with stage I and II cancer were included in the study. Patients also had to have no prior history of arthritis or other malignancy in order to be eligible. Patient identifiers were removed from the samples, and the MSKCC IRB approved the study.

Twenty-seven anonymous serum samples from normal individuals were obtained from the blood donor room at MSKCC, and 19 previously defined normals (Bast, et al. N Engl J Med (1983) 309:883-887) were selected. These individuals were not screened for pre-existing conditions such as arthritis or malignancy. Serum samples were also obtained, with IRB approval, from 61 individuals enrolled in the high-risk ovarian cancer screening program (Hensley et al. Gynecol Oncol (2003) 89:440-446). High-risk individuals were also followed with CA125 serum marker and pelvic sonogram. Serum samples tested for YKL-40 were from all individuals who had been followed for at least 1 year in the high-risk screening program without evidence of ovarian cancer. Serum samples from 33 individuals without known cancer with benign gynecologic disease such as uterine fibroids, ovarian cysts, endometriosis, and endometrial hyperplasia were identified in the MSKCC GYN tissue bank.

Blood Collection and Serum Separation

All blood samples were collected from patients with ovarian, fallopian tube, or primary peritoneal cancer 1 to 2 weeks prior to primary surgical resection of their tumors. The blood samples from patients, normal individuals, and high-risk individuals were allowed to clot at room temperature for at least 30 minutes, and were then centrifuged at 40° C., for 5 minutes at 1500 rpm. The serum aliquots were stored at −20° C. until tested.

YKL-40 ELISA Assay

YKL-40 levels were determined, in duplicate, for all serum samples, using the commercially available YKL-40 ELISA Kit from Metra Biosystems (Mountain View, Calif.), according to the manufacturer's protocol. Protein concentrations were determined as absorbances using the Biorad Benchmark Microplate Reader.

CA125 and CA15-3 Serum Analysis

CA125 and CA15-3 serum testing was performed in the clinical chemistry laboratory of MSKCC, on an Immuno 1 analyzer from Bayer Diagnostics (Tarrytown, N.Y.). For data analysis, the upper limit of normal for CA125 and CA15-3 were defined as 35 U/mL.

Statistical Analysis

To analyze the data, we divided patients into different groups according to clinical and pathological parameters. Comparisons between groups were done using Student's t-test, after applying the logarithmic transformation to reduce the skewness in the distribution of the laboratory assay results. Correlations between numeric variables were assessed by Spearman rank-correlation coefficient. YKL-40, CA125, and CA15-3 serum concentrations were also classified as normal or elevated. The statistical significance of the relative accuracy of YKL-40 versus that of either CA125 or CA15-3 in detecting cancer among subgroups of patients was based on cases where two tests gave discrepant results using McNear's test. The relationship of these dichotomous variables to other clinicopathological correlates was established with the X2 test or the Fisher's exact test, as appropriate.

Receiver operating characteristics curves (ROC) were constructed for YKL-40 and CA125 serum concentrations as diagnostics for cancer by plotting sensitivity versus 1-specificity, and the area under each ROC curve (AUC) was calculated. Statistical analysis comparing the ROC curves of YKL-40 and CA125 was performed using the method of DeLong et al. (Biometrics (1988) 44: 837-845). A Kaplan-Meier progression-free survival curve was constructed to demonstrate, in stage I and II patients, the progression-free survival difference between patients with YKL-40 elevations less than and greater than 80 ng/mL The log-rank test was used to examine the significance of the relationship between log-marker values and progression-free survival.

### EXAMPLE 1

### Normal Subject YKL-40, CA125, and CA15-3 Values

Serum was collected from 46 normals. As depicted in Table 1, the range of YKL-40 levels in the normal patients was 15-166 ng/mL. The mean and median YKL-40 values were 33.5 ng/mL and 28 ng/mL, respectively. The mean value is virtually identical to the mean serum YKL-40 value of 33 ng/mL obtained for 102 healthy women in another recent publication (Dehn et al, supra). The upper limit of normal for YKL-40 in this group of normal individuals was defined as 61 ng/mL, based on the mean value plus 2 standard deviations (95% CI). Thus, an abnormal YKL-40 serum level was determined to be 62 ng/mL. This value is consistent with the reagent vendor (Metra Biosystems, Mountain View, Calif.). Four of 46 individuals had YKL-40 values ≧62 ng/mL; these values were 166, 140, 72, and 62 ng/mL. For the sake of confidentiality, normal individuals were not questioned about a personal history of arthritis or cancer.

CA125 mean and median values were 13.4 U/mL and 11.5 U/mL (range 4-31 U/mL), respectively. CA15-3 mean and median values were 17 U/mL and 15.5 U/mL (range, 7-34 U/mL).

High-Risk Screening Patient YKL-40, CA125, and CA15-3 Values

Serum was collected from patients followed for more than 1 year in the high-risk ovarian cancer-screening program at MSKCC. Nineteen patients had no personal history of malignancy. Forty-two patients had a personal history of breast cancer for which they were without evidence of disease. All patients had a strong family history of breast or ovarian cancer (see Table 1).

The median YKL-40 values were 44.5 ng/mL (range, 5-133 ng/mL) and 36 ng/mL (range, 9-69 ng/mL) for high-risk individuals with and without a personal history of breast cancer, respectively. There was no statistically significant difference between these groups or between either high-risk group and the normal individuals tested. For the patients with no prior history of cancer, one patient had a YL-40 value of 69 ng/mL. For the patients with a prior history of breast cancer, 11 patients had YKL-40 values greater than 61 ng/mL. Of note, three of these patients, with YKL-40 values of 87 ng/mL, 87 ng/mL, and 62 ng/mL, were found 18, 8, and 18 months later, respectively, to have new pulmonary nodules, recurrent breast cancer, and an abdominal mass on a CT scan, respectively. CA125 values for all screening patients were <35 U/mL. Interestingly, there was a difference between mean CA125 values in screening patients with prior cancer and those without prior cancer (P<0.001), and between screening patients with prior cancer and normals (P<0.015).

### EXAMPLE 3

### Patients with Benign Gynecologic Processes

Individuals with benign gynecologic processes based on transvaginal sonogram and pathology reports were identified from the high-risk ovarian screening program. Thirty-three individuals were identified. Diagnoses included uterine fibroids (16), simple ovarian cysts (10), complex ovarian cysts (6), corpus luteum cysts (3), endometrial polyps (2), atypical endometrial hyperplasia (2), and endometriosis (1).

For the patients with benign gynecologic disorders, the median YKL-40 value was 38 ng/mL (range, 5-67 ng/mL), and median CA125 values was 12.5 U/mL (range, 5 to 274 U/mL) (see Table 1). There was no statistically significant difference between the YKL-40 values of this group and the high-risk groups or the normal individuals tested.

All patients in the benign gynecologic process group had YKL-40 values less than 62 ng/mL except for the two patients with atypical endometrial hyperplasia (YKL-40 values of 62 and 67 ng/mL). All patients in this group had CA125 values less than 35 U/mL except for one patient with endometriosis (CA125 value of 274 U/mL). This patient remains disease-free at 18 months follow-up.

### EXAMPLE 4

### Serum YKL-40 Levels Ovarian Cancer Patients

Preoperative serum samples from fifty epithelial ovarian cancer patients were evaluated in this study. Patient demographics are outlined in Table 2. The median age of patients was 59 (range, 31-81). Forty-six of the 50 patients (92%) had a diagnosis of primary ovarian cancer. Four patients had primary fallopian tube or peritoneal cancer. Thirty-one (62%) of 50 patients in the study had stage I or II cancers, while the rest had advanced-stage or recurrent disease. Thirty-seven (74%) of the tumors were histological grade 3. Twenty-two patients (44%) had tumors with serous histology, the most common histological tumor type. Clinical follow-up was available on 47 of the 50 patients (94%). Median long-term follow-up of 99 months (range, 33 to 125 months) was available for stage I and II tumors. Thirty-seven (74%) of the patients in the study were alive, and 30 remained in remission (60%).

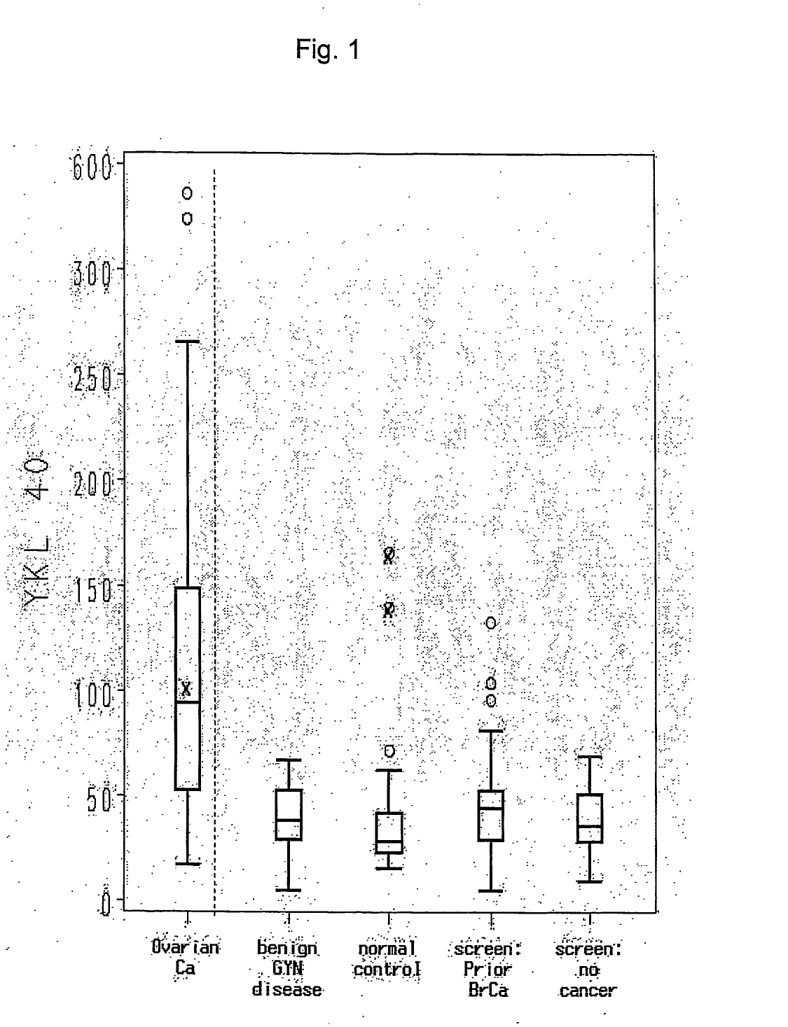

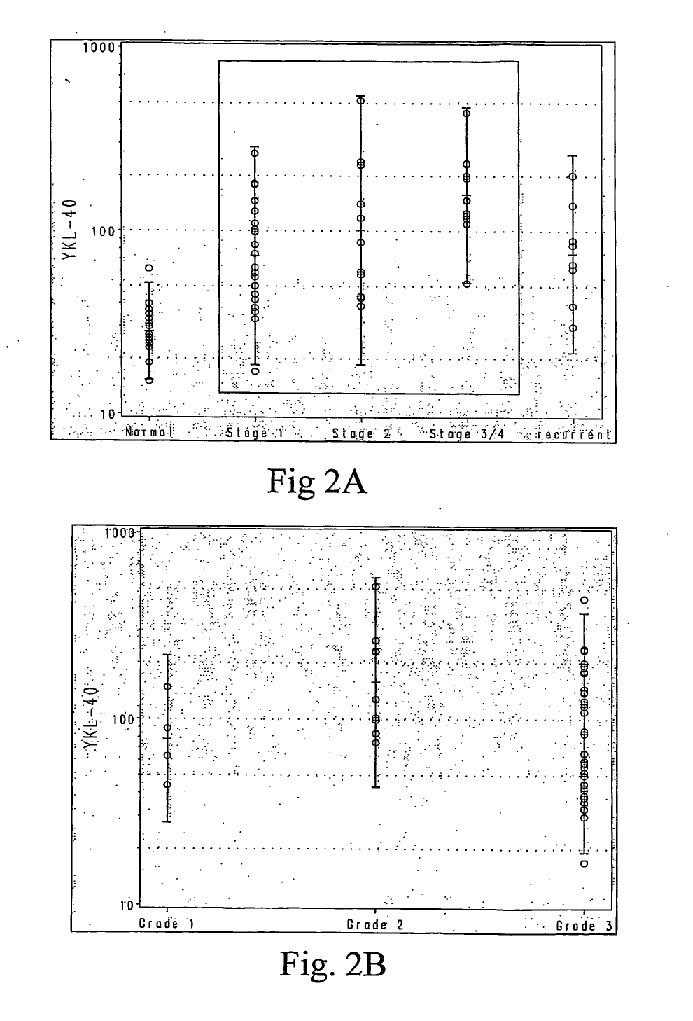

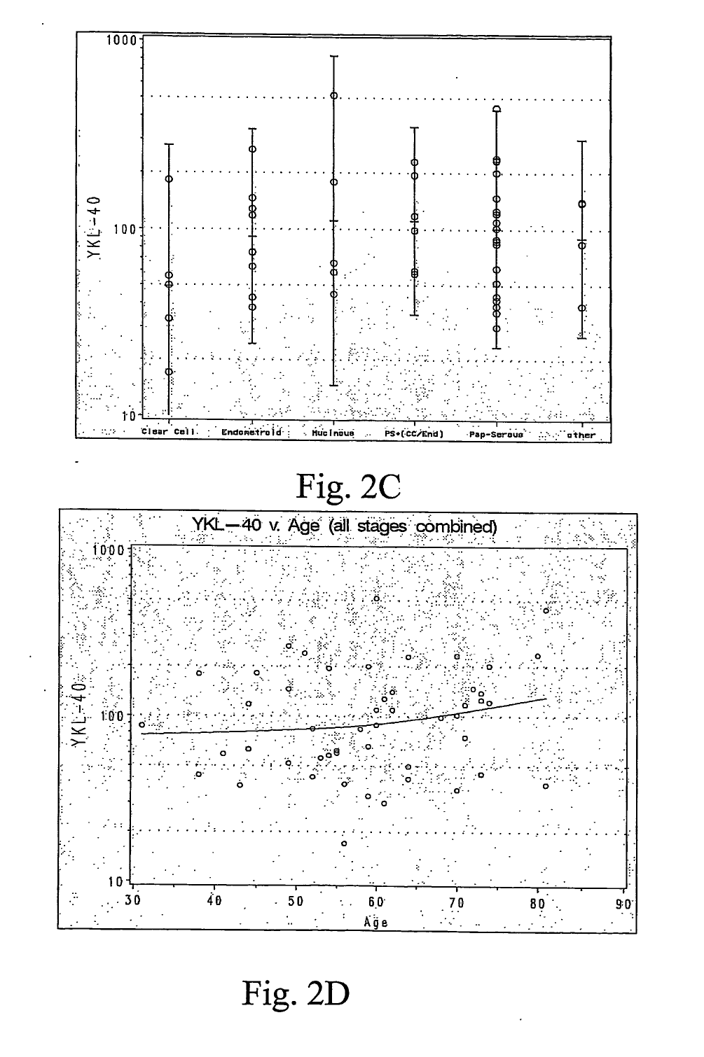

The mean and median YKL-40 levels in all epithelial ovarian cancer patients were 121.8 ng/mL and 94 ng/mL (range, 17-517 ng/mL), respectively (see Table 1). As illustrated in FIG. 1, preoperative YKL-40 levels were significantly higher in epithelial ovarian cancer patients (P<0.0001) relative to both normal controls and individuals in the high-risk epithelial ovarian cancer-screening program. Patients with stage I tumors had preoperative serum YKL-40 levels approximately 2.59 times higher than normal controls (FIG. 2A).

Serum values of YKL-40, CA125, and CA15-3 were determined for epithelial ovarian cancer patients in the study (Table 3). For all patients examined, 36 out of 50 epithelial ovarian cancer patients (72%) had elevated YKL-40 serum levels compared with 23 of 50 (46%), and 13 of 50 patients (26%) for CA125 and CA15-3, respectively. Serum YKL-40 testing was significantly better (P<0.008) than CA125 and CA15-3 (P<0.0001) at detecting epithelial ovarian cancer when considering patients for whom YKL-40 and CA125 gave discordant results. Nine patients (18%) had elevations of all three markers; 12 patients (24%) had elevations of YKL-40 and CA125; 15 patients (30%) had elevations of YKL-40 alone; one patient (2%) had an elevation in CA125 and CA15-3; two patients (4%) had an elevation in CA 125 alone; two patient (4%) had an elevation in CA15-3 alone; and nine patients (18%) did not have elevations in any of the markers.

The mean and median YKL-40 levels in stage I and II ovarian cancer patients were 109 ng/mL and 75 ng/mL, respectively (range, 17-517 ng/mL). Preoperative serum levels of YKL-40, CA125, and CA15-3 were elevated in 20 of 31 (65%), 11 of 31 (35%), and four of 31 (13%) stage I and II ovarian cancer patients, respectively. YKL-40 was significantly more likely to detect early-stage ovarian cancer than were CA125 and CA15-3 (P=0.039).

The mean and median YKL-40 levels in 11 advanced-stage ovarian cancer patients were 181 ng/mL and 148 ng/mL (range, 52445 ng/mL), respectively. The mean and median YKL-40 levels in eight recurrent patients were 88 ng/mL and 79 ng/mL (range, 30-202 ng/mL), respectively. Preoperative serum levels of YKL-40 were elevated in patients with advanced-stage, ten of 11 (91%), and recurrent, six of eight (75%), ovarian cancer (Table 3). Preoperative serum levels of CA 125 were elevated in patients with advanced and recurrent ovarian cancer: seven of 11 (64%) and five of eight (63%) for stage III/IV and recurrent tumors, respectively. Preoperative serum levels of CA15-3 were elevated in patients with advanced and recurrent ovarian cancer: six of 11 (55%) and three of eight (38%) for stage III/IV and recurrent tumors, respectively.

### EXAMPLE 5

### Frequency of Serum YKL-40 Values in Ovarian Cancer Patients as a Function of Tumor Stage, Grade Histology, and Age

Table 3 enumerates the frequency of elevation of preoperative serum levels of YKL-40, CA125, and CA15-3 in patients who were subsequently diagnosed as having primary ovarian, fallopian tube, or peritoneal cancer on surgical pathologic review. Table 3 further delineates the frequency of elevation of these three serum makers, taking into consideration primary disease site, stage, grade, and histology. The number of patients with elevated serum YKL-40 values was higher than that for CA125 and CA15-3 in all groups regardless of these variables.

As depicted in FIG. 2A, preoperative serum levels of YKL-40 increased with increasing stage of the tumor (stage I vs. II vs. III/IV by simple Spearman correlation: r=0.425, P<0.005). Patients with stage II tumors had preoperative YKL-40 values that were 1.39 times higher than those of patients with stage I tumors. Patients with stage III/IV tumors had YKL-40 values that were 1.58 times higher than those of stage II patients. There was a linear trend in YKL-40 elevation when stage I, I and III/IV patients were compared (P<0.005). Of note, patients with recurrent tumors had elevated but lower overall values of serum YKL-40 than patients with newly diagnosed stage II, III, IV tumors. Serum YKL-40 values were approximately 50% lower in patients with pure clear-cell tumors.

Serum YKL-40 values were elevated across all grades of ovarian tumors (FIG. 2B). Thirty-seven (74%) of the 50 patients in the study with ovarian tumors had grade 3 tumors; 24 (65%), 17 (46%), and 11 (30%) of these 37 patients had elevated serum YKL-40, CA125, and CA15-3 values, respectively.

Serum YKL-40 values were elevated in all histological subtypes (FIG. 2C). YKL-40 was elevated in 16 of 22 (73%) of patients with serous tumors and in six of eight (75%) of patients with endometrioid tumors. CA125 and CA15-3 were elevated in 12 of 22 (55%) and 10 of 22 (45%) patients with serous tumors, respectively. Three out of eight (38%) patients with endometrioid tumors had elevations of serum CA125, and none of these patients had elevations in CA15-3. Patients with clear-cell tumors were less likely to have elevations of YKL-40 (1 of 5, 20%), CA125 (1 or 5, 20%), and CA15-3 (0 of 5). YKL-40 was elevated in four of five patients with mucinous tumors, while serum CA125 and CA15-3 were normal in all of these patients. There was no significant correlation between YKL-40 and age overall (r=0.18, P=0.20) (FIG. 2D).

### EXAMPLE 6

### ROC Curve of YKL-40 in Ovarian Cancer

FIG. 3 depicts the ROC curves for YKL-40 and for CA125. Curves were generated for ovarian cancer patients versus normal controls, for ovarian cancer patients versus screening individuals, and for ovarian cancer versus benign gynecologic processes. The approximate area under the ROC curve assessing serum YKL-40 as a diagnostic tool for the detection of ovarian cancer against normal controls was 0.889 compared with 0.782 for CA125 (P=0.045) (FIG. 3). At the value of 61 ng/mL, Y 0 has a sensitivity and specificity of 72% and 90%, respectively, for the detection of ovarian cancer. The CA125 value of 35 U/mL, in this patient population, has a sensitivity and specificity of 47% and 95%, respectively, for the detection of ovarian cancer. The approximate area under the ROC curve for ovarian cancer patients versus screening patients was 0.817 and 0.753 for YKL-40 and CA125, respectively. The approximate area under the ROC curve for ovarian cancer patients versus benign gynecologic processes was 0.857 and 0.761 for YKL-40 and CA125, respectively.

### EXAMPLE 7

### Serum YKL-40 Levels in Stage I and II Patients and Disease-Free Survival

Thirty-one patients in the study had stage I and II tumors, and 29 of these patients had long-term follow-up (99 months, range 33-125 months) and were evaluable for disease recurrence. Of these 29 patients, 10 (34%) had recurrence of disease. All ten patients that recurred had preoperative YKL-40 values that were >80 ng/mL. In fact, of the 29 patients, 14 had YKL-40 values >80 ng/mL, and ten recurred (71%). Of fifteen patients with preoperative YKL-40 values <80 ng/mL, all remain in remission. Moreover, of the 14 patients with YKL-40 values >80 ng/mL, nine have died of ovarian cancer; and of the 15 patients with YKL-40 values <80 ng/mL, none has died of ovarian cancer.

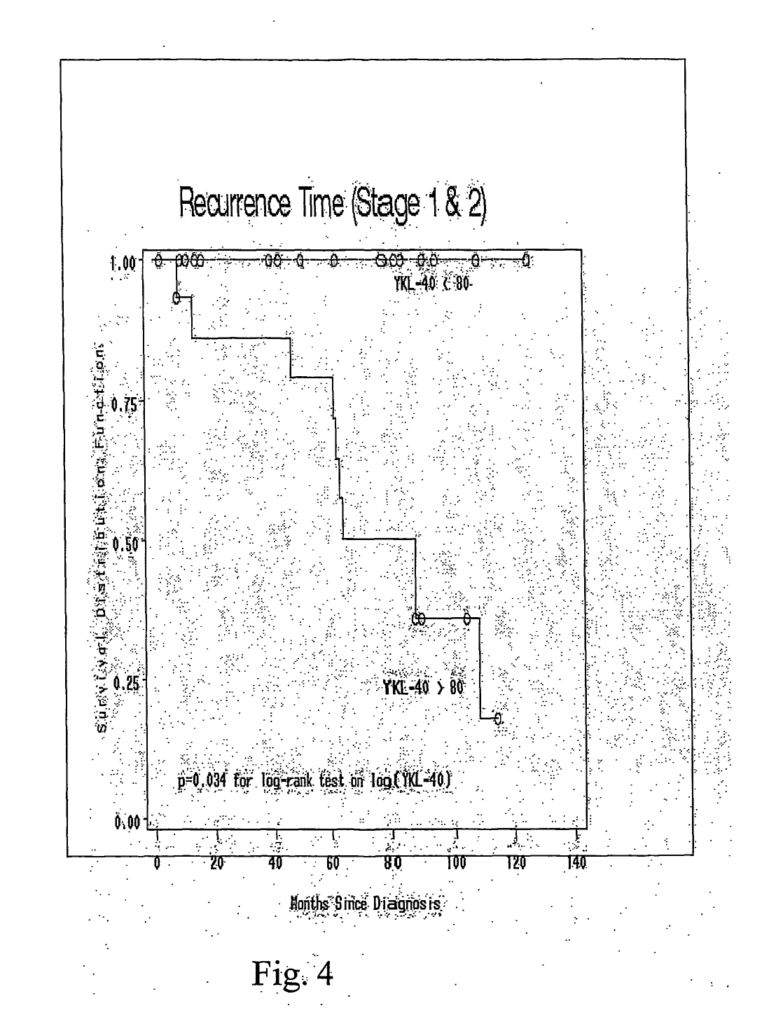

FIG. 4 depicts the recurrence-free survival curves for patients with stage I and II ovarian cancer. The two curves illustrate that patients who had preoperative serum YKL-40 values that were >80 ng/mL had shorter disease-free survival than patients with YKL-40 values <80 ng/mL (P=0.034 based on the log-rank test for log-YKL-40 as a continuous-valued predictor). Neither CA125 nor CA15-3 was predictive of recurrence in this patient population.

TABLE 1
Serum YKL-40, CA125, and CA15-3 Values for Normal, High-Risk Screening
Population, Benign Gynecologic Disorder, and Ovarian Cancer Patients

| | | High-Risk | High-Risk | Benign | |
|---|---|---|---|---|---|
| | | Screening | Screening | Gynecologic | Ovarian |
| | Normal | (No Cancer) | (Prior Cancer) | Disease | Cancer |
| Number of Individuals | 46 | 19 | 42 | 33 | 50 |
| Mean YKL-40 Value (ng/mL) | 33.5** | 38** | 44.4** | 39.6** | 121.8 |
| Median YKL-40 Value (ng/mL) | 28 | 36 | 44.5 | 38 | 94 |
| Range of YKL-40 Values (ng/mL) | 15-166 | 9-69 | 5-133 | 5-67 | 17-517 |
| 1 Standard Deviation | 13.7 | 12.1 | 19.1 | 15.3 | 99.2 |
| Mean YKL-40 + 2 SD (ng/mL) | 61 | 62 | 82 | 70 | 320.2 |
| Mean CA125 Value (U/mL) | 13.4* | 10.8* | 16.9* | 21* | 272 |
| Median CA125 Value (U/mL) | 11.5 | 7.0 | 15.5 | 12.5 | 30 |
| Range of CA125 Values (U/mL) | 4-31 | 5-26 | 5-33 | 5-274 | 5-2460 |
| Mean CA15-3 Values (U/mL) | 17 | NP | NP | NP | 41.5 |
| Median CA15-3 Value (U/mL) | 15.5 | NP | NP | NP | 23 |
| Range of CA15-3 Values (U/mL) | 7-34 | NP | NP | NP | 5-470 |

NP = Not Performed
*P < 0.001 for Screening Patients with Prior Breast Cancer vs Screening Patients with No Cancer; P = 0.015 for Screening Patients with Prior Breast Cancer vs Normals; based on log (CA125)
**P value Not Significant for Prior Cancer vs No Cancer, for Prior Cancer vs Normal, or for Benign Gynecologic Disease vs Normal based on log (YKL-40)

TABLE 2
Epithelial Ovarian Cancer Patient Demographics

| | Number | |
|---|---|---|
| All Patients | 50 | |
| Age (Range) | 59 | (31-81) |
| Primary Disease Site | | |
| Ovarian | 46 | (92%) |
| Fallopian Tube | 3 | (6%) |
| Peritoneal | 1 | (2%) |
| Stage | | |
| I | 20 | (40%) |
| II | 11 | (22%) |
| III/IV | 11 | (22%) |
| Recurrent | 8 | (16%) |
| Grade | | |
| 1 | 4 | (8%) |
| 2 | 9 | (18%) |
| 3 | 37 | (74%) |
| Histological Diagnosis | | |
| Serous | 22 | (44%) |
| Endometroid | 8 | (16%) |
| Mucinous | 5 | (10%) |
| Clear Cell | 5 | (10%) |
| Other | 10 | (20%) |
| Patient Status | | |
| NED | 30 | (60%) |
| Recurred | 17 | (34%) |
| Living | 37 | (74%) |
| Deceased | 10 | (20%) |
| Unknown | 3 | (6%) |

TABLE 3
Preoperative Serum YKL-40, CA125, and CA15-3
Values for Ovarian Cancer Patients

| | | | | | P-Value** | | |
|---|---|---|---|---|---|---|---|
| | | | | | for | | |
| | Elevated YKL-40 | | Elevated CA125 | | YKL-40 | Elevated CA15-3 | |
| | (≧62 ng/mL) | | (>35 U/mL) | | vs. CA125 | (>35 U/mL) | |
| All Patients | 36/50 | (72%) | 23/50 | (46%) | 0.008 | 13/50 | (26%) |
| Primary Disease Site | | | | | | | |
| Ovarian | 32/46 | (70%) | 20/46 | (43%) | 0.013 | 11/46 | (24%) |
| Fallopian Tube | 3/3 | (100%) | 2/3 | (67%) | 1.0 | 1/3 | (33%) |
| Peritoneal | 1/1 | (100%) | 1/1 | (100%) | 1.0 | 1/1 | (100%) |
| Stage | | | | | | | |
| I/II | 20/31 | (65%) | 11/31 | (35%) | 0.039 | 4/31 | (13%) |
| III/IV | 10/11 | (91%) | 7/11 | (64%) | 0.25 | 6/11 | (55%) |
| Recurrent | 6/8 | (75%) | 5/8 | (63%) | 1.0 | 3/8 | (38%) |
| Tumor Grade | | | | | | | |
| 1 | 3/4 | (75%) | 2/4 | (50%) | 1.0 | 0/4 | |
| 2 | 9/9 | (100%) | 4/9 | (44%) | 0.063 | 2/9 | (22%) |
| 3 | 24/37 | (65%) | 17/37 | (46%) | 0.109 | 11/37 | (30%) |
| Histological Diagnosis | | | | | | | |
| Serous | 16/22 | (73%) | 12/22 | (55%) | 0.219 | 10/22 | (45%) |
| Endometroid | 6/8 | (75%) | 3/8 | (38%) | 0.125 | 0/8 | |
| Mucinous | 4/5 | (80%) | 0/5 | | 0.125 | 0/5 | |
| Clear Cell | 1/5 | (20%) | 1/5 | (20%) | 1.0 | 0/5 | |
| Other* | 9/10 | (90%) | 7/10 | (70%) | 1.0 | 3/10 | (30%) |

*Mixed Histology (e.g., Clear Cell/Papillary Serous, Adenocarcinoma, Poorly Differentiated, Papillary Serous/Endometroid)
**Based on McNemar's test for discordant pairs.
NS = Not Significant

1. A method for detection of ovarian cancer in an individual who has not been diagnosed with ovarian cancer and who is not displaying symptoms associated with stage III or IV ovarian cancer, comprising the steps of
(a) obtaining a sample from the individual;
(b) determining the amount of expressed YKL-40 in the sample; and
(c) comparing the amount of expressed YKL-40 determined to a pre-determined threshold, wherein if the predetermined threshold is exceeded, the test is deemed to be an indicator of ovarian cancer in the individual.

2. The method of claim 1, wherein the sample is a serum or plasma sample.

3. The method of claim 2, wherein the pre-determined threshold is at least two standard deviations higher than the mean amount of YKL-40 detected in a population of individuals who do not have ovarian cancer.

4. The method of claim 3, wherein the population of individuals is composed of individuals within an age group range of which the individual tested is a member.

5. The method of claim 3, wherein the population of individuals consists of individuals who themselves have increased risk factors for ovarian cancer.

6. The method of claim 1, wherein the pre-determined threshold is at least two standard deviations higher than the mean amount of YKL-40 detected in a population of individuals who do not have ovarian cancer.

7. The method of claim 6, wherein the population of individuals is composed of individuals within an age group range of which the individual tested is a member.

8. The method of claim 6, wherein the population of individuals consists of individuals who themselves have increased risk factors for ovarian cancer.

9. The method of claim 1, wherein the amount of YKL-40 is determined using an immunoassay.

10. The method of claim 1, further comprising the step of testing the sample for the amount of CA-125 and taking both the measured amount of YKL-40 and the measured amount of CA-125 into consideration in determining the presence of ovarian cancer.

11. A method for assessing the risk of post-treatment recurrence in a patient diagnosed with early stage ovarian cancer comprising the steps of
(a) obtaining a pre-operative sample from the individual and
(b) determining the amount of expressed YKL-40 in the sample; wherein a pre-operative level of greater than 80 ng/mL is indicative of an increased risk of post-treatment recurrence of ovarian cancer.

12. The method of claim 11, wherein the sample is a serum or plasma sample.

13. The method of claim 12, wherein the amount of YKL-40 is determined using an immunoassay.

14. The method of claim 11, wherein the amount of YKL-40 is determined using an immunoassay.

15-16. (canceled)
